(19) United States
(12) Patent Application Publication
(10) Pub. No.: US 2024/0058755 A1
(43) Pub. Date: Feb. 22, 2024

(54) CONTINUOUS ENZYMATIC PERFUSION REACTOR SYSTEM

(71) Applicant: Graphium Biosciences, Inc., Cleveland, OH (US)

(72) Inventors: Tyrel R. DEUTSCHER, Wauwatosa, WI (US); Brandon J. ZIPP, Roseville, CA (US); Janee' M. HARDMAN, Fair Oaks, CA (US)

(73) Assignee: Graphium Biosciences, Inc., Cleveland, OH (US)

(21) Appl. No.: 18/265,838

(22) PCT Filed: Dec. 13, 2021
(86) PCT No.: PCT/US2021/063158

§ 371 Date: Jun. 7, 2023

Related U.S. Application Data
(60) Provisional application No. 63/124,299, filed on Dec. 11, 2020.

(51) Int. Cl. B01D 61/14; B01D 15/32; B01D 61/18; B01D 63/02; B01D 63/10; B01D 71/68; C12P 19/60

(52) U.S. Cl. CPC B01D 61/145; B01D 15/325; B01D 61/18; B01D 63/02; B01D 63/10; B01D 71/68; C12P 19/60; B01D 2311/06; B01D 2311/08; B01D 2315/10

(57) ABSTRACT

The present disclosure relates to methods, systems, and apparatus for efficient continuous production of hydrophobic products with the simultaneous retention of substrates.

### CROSS-REFERENCE TO RELATED APPLICATIONS

This application claims the benefit of priority to U.S. Provisional Patent Application No. 63/124,299, filed on Dec. 11, 2020, which is incorporated herein by reference in its entirety.

### FIELD OF THE DISCLOSURE

The present disclosure generally relates to biocatalytic reactions of hydrophobic chemistries. More specifically, the present disclosure relates to methods, systems, and apparatus for efficient continuous production of hydrophobic products with the simultaneous retention of substrates.

### SUMMARY OF THE TECHNOLOGY

Disclosed herein, are processes, systems, and apparatus for biocatalytic reactions of hydrophobic chemistries.

The disclosures relate to methods, systems, and apparatus for the efficient continuous production of hydrophobic products with the simultaneous retention of substrate.

The disclosures herein have the advantage that improved production of the desired end product is achieved. For example, a continuous biocatalytic reaction occurs in an enzymatic perfusion reactor, where a precursor molecule is converted to a glycoside, which allows for increased yields, reduced waste/workload, and the ability to produce a compound that otherwise would not have been economically feasible. The disclosures allow for the simultaneous retention of substrate while harvesting the product and the inclusion of a downstream processing step during product generation. Thus, the disclosure processes, systems, and apparatus allow for industrial production using an enzyme and catalytic reaction that would otherwise be considered inefficient as an industrial biocatalyst.

In certain embodiments, the present disclosure provides for methods of producing cannabinoid-glycosides. In some embodiments, methods herein for the production of a cannabinoid-glycoside may comprise: (a) producing the cannabinoid-glycoside in an aqueous solution to obtain a biocatalytic reaction mixture; (b) filtering the biocatalytic reaction mixture comprising the cannabinoid-glycoside to achieve a filtration retentate and a filtration permeate comprising the cannabinoid-glycoside; (c) returning the filtration retentate to a first vessel; (d) separating the cannabinoid-glycosides from the filtration permeate using reverse-phase 8 chromatography such that the cannabinoid-glycosides bind to the chromatography resin; and (e) returning unbound filtration permeate to the first vessel. In accordance with embodiments herein, a cannabinoid-glycoside herein may be a Δ9-tetrahydrocannabinol (THC)-glycoside, a cannabidiol (CBD)-glycoside, or any combination thereof.

In some embodiments, the chromatography resin is a C18 chromatography resin.

In some embodiments, a cannabinoid-glycoside herein may be a THC-glycoside and may further comprise adding THC and one or more reagents to the first vessel. In some embodiments, a cannabinoid-glycoside herein may be a CBD-glycoside and may further comprise adding CBD and one or more reagents to the first vessel. In accordance with these embodiments, the one or more reagents herein may comprise glycosyltransferase enzyme (UGT76G1), Uridine diphosphate glucose (UDP-glucose), Sucrose Synthase (SUS), sucrose, and any combination thereof. In some embodiments, a Sucrose Synthase herein may be isoform 1. In some embodiments, a Sucrose Synthase isoform 1 herein may be from Stevia rebaudiana (SrSUS1).

In some embodiments, methods herein comprising filtering may comprise modified PES (mPES). In some embodiments, filtering herein may comprise hollow-fiber tangential-flow ultrafiltration. In some embodiments, filtering herein may comprise spiral-wound configuration.

In some embodiments, methods herein may further comprise collecting cannabinoid-glycosides from the chromatography resin. In accordance with these embodiments, the chromatography resin matrix may have a large particle size.

In some embodiments, methods herein may further comprise adding THC or CBD to the first reaction vessel, wherein the THC or CBD is added to maintain a constant THC or CBD concentration throughout the entirety of the reaction. In some embodiments, methods herein may be run continuously or semi-continuously for days, weeks, or months.

In certain embodiments, the present disclosure provides systems for production of hydrophobic products. In some embodiments, a system for production of hydrophobic products may comprise: a reaction vessel having a vessel outlet and a vessel return; a reaction flow path extending from the vessel outlet to the vessel return; a precursor of the hydrophobic product to the vessel or the reaction flow path; and a filtering system disposed within the reaction flow path for separating products from the reaction flow path to achieve a filtration retentate and a filtration permeate, wherein the filtration permeate includes product, the filtering system having a filter inlet, a filter return to return the filtration retentate to the reaction flow path, and a permeate outlet for the filtration permeate.

In some embodiments, systems herein may further comprise a chromatography system for separating the product from the filtration permeate using reverse-phase chromatography such that the product binds to the chromatography resin. In some embodiments, chromatography system herein may further include a chromatography return to the vessel or the reaction flow path for unbound filtration permeate. In some embodiments, the chromatography resin is a C18 chromatography resin.

In some embodiments, systems herein may be continuous or semi-continuous. In some embodiments, a filtering system herein may include a hollow fiber PES membrane filter. In some embodiments, a reaction vessel herein may be an enzymatic perfusion reactor.

In some embodiments, systems herein may have a hydrophobic product that is a Δ9-tetrahydrocannabinol (THC)-glycoside, a cannabidiol (CBD)-glycoside, or any combination thereof. In some embodiments, systems herein may have a hydrophobic product that is a Δ9-tetrahydrocannabinol (THC)-glycoside and the precursor to the hydrophobic product is Δ9-tetrahydrocannabinol (THC) or the hydrophobic product is a cannabidiol (CBD)-glycoside and the hydrophobic product is cannabidiol (CBD).

In certain embodiments, the present disclosure provides for processes for the production of hydrophobic products. In some embodiments, a process for the production of hydrophobic products herein may comprise: (a) mixing a biocatalytic reaction mixture in a reaction system including a reaction vessel having a vessel outlet and a vessel return and a reaction flow path extending from the vessel outlet to the vessel return; (b) along the reaction flow path, filtering the biocatalytic reaction mixture to produce a filtration retentate and a filtration permeate, wherein the filtration permeate comprises the product; and (c) returning the filtration retentate to the reaction vessel.

In some embodiments, a process for the production of hydrophobic products herein may further comprise separating the hydrophobic product from the filtration permeate using reverse-phase chromatography such that the hydrophobic product binds to the chromatography resin. In some embodiments, the chromatography resin is a C18 chromatography resin. In some embodiments, a process for the production of hydrophobic products herein may further comprise returning unbound filtration permeate to the reaction vessel.

In some embodiments, a process for the production of hydrophobic products herein may be continuous or semi-continuous. In some embodiments, the filtering during a process herein may include using a hollow fiber PES membrane filter. In some embodiments, the reaction vessel of a process herein may be an enzymatic perfusion reactor.

In some embodiments, the hydrophobic product of a process herein may be a Δ9-tetrahydrocannabinol (THC)-glycoside, a cannabidiol (CBD)-glycoside, or any combination thereof. In some embodiments, the hydrophobic product of a process herein may be a Δ9-tetrahydrocannabinol (THC)-glycoside and the precursor to the hydrophobic product is Δ9-tetrahydrocannabinol (THC) or the hydrophobic product is a cannabidiol (CBD)-glycoside and the precursor to the hydrophobic product is cannabidiol (CBD).

### BRIEF DESCRIPTION OF THE DRAWINGS

The foregoing aspects and other features of the disclosure are explained in the following description, taken in connection with the accompanying drawings, wherein:

FIG. 1 is a schematic overview of the nested loops of a biocatalytic reaction in accordance with embodiments of the present disclosure.

FIG. 2 depicts the enzymatic glycosylation of THC by SrUGT76G1 coupled to UDPG recycling by SrSUS1 in accordance with embodiments of the present disclosure.

FIG. 3A is a schematic view of an exemplary reactor system and reaction flow path in accordance with embodiments of the present disclosure. The exemplary schematic illustrates the reaction taking place in a large cylindroconical stainless steel vessel and being pumped through hollow fiber mPES membranes. The filter permeate is then passed through a C18 chromatography column, and both the filter retentate and unbound permeate are returned to the reaction, removing only the reaction products.

FIG. 3B is a technical reactor schematic showing the main reactor and supporting systems including valves, pumps, filters, plumbing, jacket heater, cover gas system, sterilize in place system, substrate feed system, and various sensors and gauges.

FIG. 3C illustrates a needle through which the substrate solution is fed to encourage quick dispersion in accordance with embodiments of the present disclosure.

FIG. 3D illustrates how the substrate solution is fed in DMSO through the needle avoids precipitation or caking.

FIG. 4 is a HPLC linetrace comparing mPES filter chemistry rejection of THC and hydrophobic compounds. Solid line represents PES filter permeate. Dashed line represents mPES filter permeate.

FIG. 5 is a graph depicting the reaction yield (g/L) over time (hours). The reaction was run in semi-continuous mode with intermittent product removal. The reaction is allowed to accumulate product without filtration until the concentration of product starts to inhibit the enzyme. Filtration and removal of the product is initiated and run until the product concentration is below a pre-determined threshold or at a product concentration conducive to enzyme activity. The reaction is terminated after the rate of product formation no longer meets production specifications. The solid lines represent the accumulation phase and the broken lines the product removal phase.

FIG. 6 is a graph comparing THC-glycoside products from SrUGT76G1/SrSUS1 biocatalytic reactions. A static/batch reaction (open circles) produced 0.2 g/L total THC-glycosides over the duration of the batch. A continuous reaction (grey diamonds) produced over 1 g/L total THC-glycosides over the duration of the reaction.

FIG. 7 is a graph comparing the final THC-glycoside production in grams/Liter of static/batch vs continuous perfusion reactions with SrUGT76G1 as the biocatalyst and similar reaction recipes.

FIGS. 8A-8C show raw feed rate data of three separate 300 L reactions. FIG. 8A shows 300 L HF.6 THC feed rate raw data. FIG. 8B shows 300 L HF.7 THC feed rate raw data. FIG. 8C shows 300 L HF.5 THC feed rate raw data.

FIG. 9A shows the corrected THC feed rates of the three 300 L reactions. FIG. 9B shows the three data sets combined and fit as an exponential decay.

FIG. 10 shows comparison of efficiency of a 10 L cannabidiol (CBD) to a 300 L THC conversion reaction.

### DETAILED DESCRIPTION

Unless otherwise required by context, singular terms as used herein and, in the claims, shall include pluralities and plural terms shall include the singular.

The use of “or” means “and/or” unless stated otherwise. Furthermore, the use of the terms “comprising,” “having,” “including,” as well as other forms, such as “includes” and “included,” are intended to be inclusive and mean that there may be additional elements other than the listed elements. Also, terms such as “element” or “component” encompass both elements and components comprising one unit and elements and components that comprise more than one subunit unless specifically stated otherwise.

Disclosed herein, are processes, systems, and apparatus for biocatalytic reactions of hydrophobic chemistries. These disclosures have application to a number of biocatalytic reactions of hydrophobic chemistries. By way of non-limiting examples, the disclosures can be used for the production of hydrophobic small molecules.

The hydrophobic small molecules can be cannabinoids, terpenoids, vanillioids, phenols, polyphenols, flavonoids, steroids, thiols, and lipids.

In some embodiments the disclosures can be used for the production of glycosides. The glycosides may be cannabinoid-glycosides. The cannabinoid-glycoside may be a glycoside of a cannabinoid selected from Δ9-tetrahydrocannabinod (THC), cannabidiol (CBD), cannibichromene (CBC), cannabigerol (CBG), cannabinol (CBN), cannabichromevarin (CBCV), cannabichromevarin (CBCV), cannabidiphorol (CBDP), cannabielsoin (CBE), cannabigerol (CBG), Cannabicyclol (CBL), Cannabinol (CBN), cannabicitran (CBT), cannabigerovarin (CBGV), cannabigerol monomethyl ether (CBGM), Cannabivarin (CBV), delta-8-tetrahydrocannabinol (delta-8-THC, Δ8-THC), (−)-trans-Δ9-tetrahydrocannabiphorol (Δ9-THCP, (C7)-Δ9-THC, and THC-Heptyl), Δ9-tetrahydrocannabiorcol (Δ9-THCC, (C1)-Δ9-THC), tetrahydrocannabivarin (THCV, THV), dimethylheptylpyran, parahexyl, or any combination thereof.

The cannabinoid glycosides may be Δ9-tetrahydrocannabinod (THC)-glycosides. The cannabinoid glycosides may be cannabidiol (CBD)-glycosides.

In some embodiments, the cannabinoid-glycosides are endocannabinoid-glycosides. The endocannabinoid-glycoside may be a glycoside of a cannabinoid selected from anandamide (AEA), 2-arachidonoyl glycerol (2AG), 1-arachidonoyl glycerol (1AG), synaptamide (DHEA), palmitoyl ethanolomide (PEA), or any combination thereof.

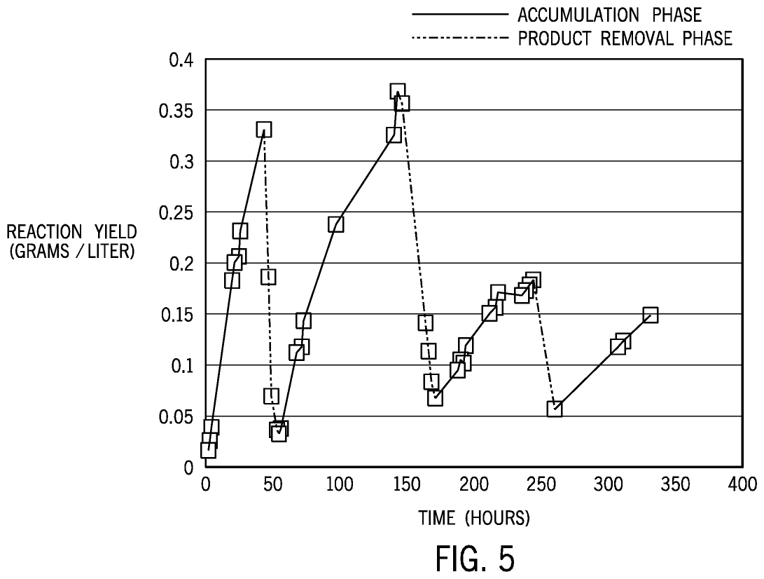

The disclosed methods, systems, and apparatus herein have the advantage that improved production of the desired product is achieved. For example, with respect to THC-glycosides and/or CBD-glycosides, a continuous biocatalytic reaction occurs in an enzymatic perfusion reactor, where THC is converted to glycosides of THC, which allows for increased yields, reduced waste/workload, and the ability to produce a compound that otherwise would not have been economically feasible. An example of the increased yields of THC-glycosides obtained from the disclosed systems and process can be seen in FIGS. 5-7. Similarly, by way of another example, with respect to CBD-glycosides, a continuous biocatalytic reaction occurs in an enzymatic perfusion reactor, where CBD is converted to glycosides of CBD. The disclosures allow for the simultaneous retention of substrate while harvesting the product and the inclusion of a downstream processing step during product generation. Thus, the disclosure processes, systems, and apparatus allow for industrial production using an enzyme and catalytic reaction that would otherwise be considered inefficient as an industrial biocatalyst.

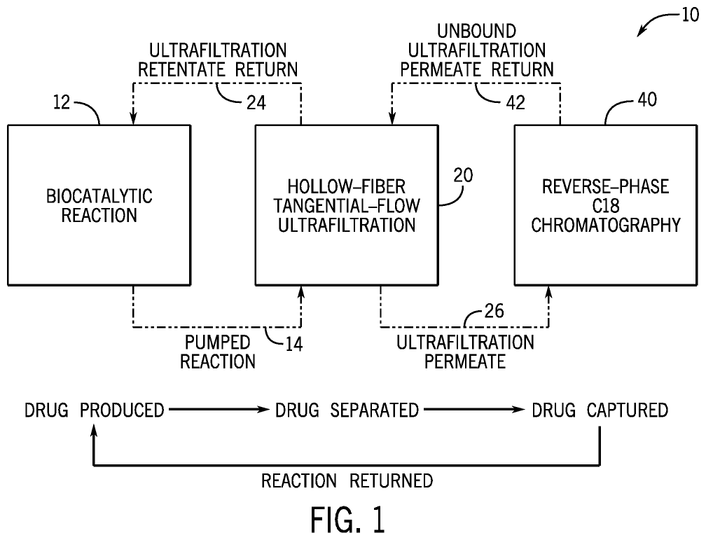

A non-limiting schematic overview of the disclosed processes is provided in FIG. 1. Briefly, a biocatalytic reaction occurs in the leftmost box (12). The reaction is pumped to the second box (20), where it is filtered with a majority of the reaction returning to the first box (12). The small amount that permeates through the filter is pumped to the third box (40). Reaction products are bound to the C18 chromatography resin, and unbound materials are returned to the reaction. The reaction is stripped of products that would inhibit the biocatalyst, freeing up further catalysis of more product while also acting as an intra-reaction processing step.

The disclosure provides a process for the production of hydrophobic molecules (products). The process may comprise: (a) producing the desired product in an aqueous solution to obtain a biocatalytic reaction mixture; (b) filtering the biocatalytic reaction mixture comprising the desired product to achieve a filtration retentate and a filtration permeate comprising the desired product; (c) returning the filtration retentate to a first vessel; (d) separating the desired product from the filtration permeate using reverse-phase chromatography such that the desired product binds to the chromatography resin; and (e) returning unbound filtration permeate to the first vessel. In some embodiments, the process further comprises adding aprecursor to the product and one or more reagents to the first vessel. The chromatography resin is a hydrophobic reverse-phase resin. The resin may be C18 resin, a C8 resin, a phenyl resin, or other hydrophobic ligands linked to silica or suitable chromatography support matrix.

In some embodiments, the disclosure provides a process for the production of cannabinoid-glycosides through biocatalytic glycosylation. The process may comprise: (a) producing a cannabinoid-glycoside in an aqueous solution to obtain a biocatalytic reaction mixture; (b) filtering the biocatalytic reaction mixture comprising the cannabinoid-glycoside to achieve a filtration retentate and a filtration permeate comprising the cannabinoid-glycoside; (c) returning the filtration retentate to a first vessel; (d) separating the cannabinoid-glycoside from the filtration permeate using reverse-phase chromatography such that the cannabinoid-glycoside bind to the chromatography resin; and (e) returning unbound filtration permeate to the first vessel. In some embodiments, the process further comprises adding a cannabinoid aglycone (precursor to the cannabinoid-glycoside) and one or more reagents to the first vessel. In some embodiments the one or more reagents comprise the cannabinoid, glycosyltransferase enzyme (UGT76G1), uridine diphosphate glucose (UDP-glucose), sucrose synthase, sucrose, and any combination thereof. In some embodiments the Sucrose Synthase is isoform 1 (SUS1). In further embodiments, the Sucrose Synthase is isoform 1 from Stevia rebaudiana (SrSUS1).

In some instances, when the desired product is a cannabinoid-glycoside, the precursor to the desired cannabinoid-glycoside may be a cannabinoid. As will be appreciated by those of skill in the art, the cannabinoid precursor may, in certain instances, be a cannabinoid-glycoside that is further glycosylated. In other instances, the precursor may be a cannabinoid that is not glycosylated.

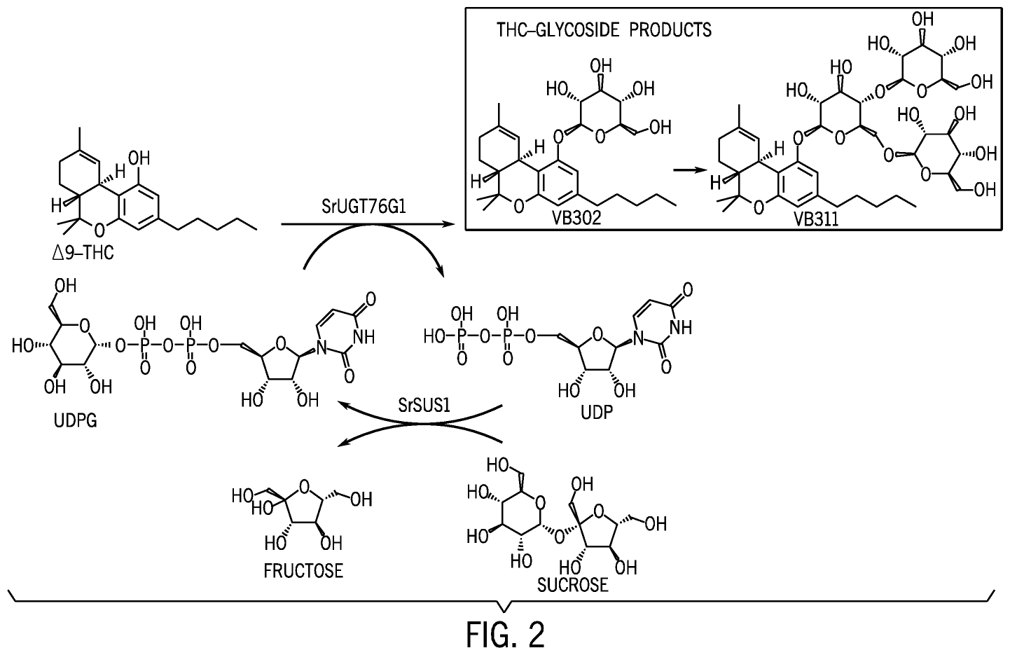

Referring to FIGS. 1 and 2, in another embodiment, the disclosure is related to a process for the production of Δ9-tetrahydrocannabinol (THC)-glycoside through biocatalytic glycosylation. The process may comprise: (a) producing the THC-glycoside in an aqueous solution to obtain a biocatalytic reaction mixture by reacting the mixture in a reaction vessel; (b) filtering the biocatalytic reaction mixture comprising the THC-glycoside to achieve a filtration retentate and a filtration permeate comprising the THC-glycoside; (c) returning the filtration retentate to the reaction vessel; (d) separating the THC-glycosides from the filtration permeate using reverse-phase C18 chromatography such that the THC-glycosides bind to the C18 chromatography resin; and (e) returning unbound filtration permeate to the reaction vessel. In some embodiments, the process further comprises adding THC (precursor to the THC-glycoside) and one or more reagents to the first vessel. In some embodiments the one or more reagents comprise THC, glycosyltransferase enzyme (UGT76G1), uridine diphosphate glucose (UDP-glucose), Sucrose Synthase, sucrose, and any combination thereof. In some embodiments the Sucrose Synthase is isoform 1 (SUS1). In further embodiments, the Sucrose Synthase is isoform 1 from Stevia rebaudiana (SrSUS1).

In another embodiment, the disclosure is related to a process for the production of cannabidiol (CBD)-glycoside through biocatalytic glycosylation. The process may comprise: (a) producing the CBD-glycoside in an aqueous solution to obtain a biocatalytic reaction mixture; (b) filtering the biocatalytic reaction mixture comprising the CBD-glycoside to achieve a filtration retentate and a filtration permeate comprising the CBD-glycoside; (c) returning the filtration retentate to a first vessel; (d) separating the CBD-glycosides from the filtration permeate using reverse-phase C18 chromatography such that the CBD-glycosides bind to the C18 chromatography resin; and (e) returning unbound filtration permeate to the first vessel. In some embodiments, the process further comprises adding CBD (precursor to the CBD-glycoside) and one or more reagents to the first vessel. In some embodiments, the one or more reagents comprise CBD, glycosyltransferase enzyme (UGT76G1), uridine diphosphate glucose (UDP-glucose), sucrose synthase, sucrose, and any combination thereof. In some embodiments, the Sucrose Synthase is isoform 1 (SUS1). In further embodiments, the Sucrose Synthase is isoform 1 from Stevia rebaudiana (SrSUS1).

Referring to FIG. 1, a schematic overview of the nested loops of a biocatalytic reaction 10 in accordance with embodiments of the present disclosure is provided. A reaction stage biocatalytic reaction occurs, as indicated by box 12. The contents of the reaction are pumped via line 14 to a filtering stage, as indicated by box 20, where the contents are filtered with a majority of the reaction returning to the reaction stage (box 12). In the illustrated embodiment, the filtering stage includes hollow-fiber tangential-flow ultrafiltration. The filtration retentate is returned from the filtering state (box 20) to the reaction stage (box 12). The small amount of filtration permeate from the filtering stage (box 20) is pumped via line 26 to the product removal stage, as indicated by box 40. In the illustrated embodiment, the product removal phase includes reverse phase C18 chromatography, however other hydrophobic reverse-phase resins may be used and can be selected by those of skill in the art. In the product removal phase (box 40), reaction products are bound to the chromatography resin, and unbound materials are returned to the reaction via line 42. In the product removal stage (box 40), the reaction is stripped of the products (cannabinoid-glycosides) that would inhibit the biocatalyst, freeing up further catalysis of more product while also acting as an intra-reaction processing step.

In some embodiments, the filtering is hollow-fiber tangential-flow ultrafiltration. In some embodiments, the filtration chemistry is implemented in a spiral-wound configuration. In some embodiments, the tangential-flow chemistry is made up of modified polyethersulfone (mPES, Repligen Corp.) that rejects hydrophobic compounds while allowing polar compounds to pass.

In some embodiments, the process further comprises collecting the products (such as cannabinoid-glycosides) from the chromatography resin. The high concentration of sugar in the reaction may be very viscous and may clog smaller particle size resin. In some embodiments, the chromatography matrix may comprise a large particle size to allow for the high concentration of sugar to pass through and not foul the chromatography resin bed. In some embodiments, the particle size of the matrix is 90-130 μm.

The products (such as cannabinoid-glycosides) can be collected by removing the “loaded” or “bound” columns from the reactor and removing the product from the columns. In some instances, the columns can be swapped out with columns that do not contain “loaded” or “bound” product. The “loaded” or “bound” columns can then be processed offline by washing the product off. The product may be washed off with ethanol or a suitable elution solvent.

The products can be produced in an aqueous solution using a biocatalytic reaction to create a biocatalytic reaction mixture. In the example of the production of cannabinoid-glycosides, the biocatalytic reaction mixture may comprise cannabinoid-glycosides and one or more reagents for the biocatalytic reaction. The first reaction vessel may comprise one or more reagents for the biocatalytic reaction. The one or more reagents may comprise a cannabinoid aglycone, glycosyltransferase enzyme (UGT76G1), Uridine diphosphate glucose (UDP-glucose), Sucrose Synthase, sucrose, or any combination thereof. The Sucrose Synthase can be one or more of isoforms 1 through 6. The Sucrose Synthase can be isoform 1 (SUS1). The Sucrose Synthase can be from any plant species. In some embodiments, the Sucrose Synthase is from Arabidopsis thaliana or Stevia rebaudiana. In some embodiments, the Sucrose Synthase is isoform 1 from Stevia rebaudiana (SrSUS1). The aqueous solution may comprise one or more reagents for the biocatalytic reaction. The one or more reagents may comprise the cannabinoid, glycosyltransferase enzyme (UGT76G1), Uridine diphosphate glucose (UDP-glucose), Sucrose Synthase (SUS), sucrose, and any combination thereof. With regards to the biocatalytic reaction, briefly, a recombinant glycosyltransferase enzyme (SrUGT76G1) covalently links glucose molecules to the cannabinoid (for example THC or CBD). Uridine diphosphate glucose (UDP-glucose) acts as the sugar donor and is regenerated within the reaction using a second recombinant enzyme, sucrose synthase (SrSUS1). SrSUS1 catalyzes the splitting of sucrose to fructose and glucose to recycle UDP into UDP-glucose (FIG. 1).

As the products (e.g., cannabinoid-glycosides) are eventually removed from the biocatalytic reaction mixture as disclosed herein, to maintain a constant precursor (e.g., cannabinoid aglycone) concentration throughout the entirety of the reaction, the precursor (e.g., cannabinoid aglycone) may be added to the aqueous solution. In some embodiments, the process further comprises the addition of the precursor into the first reaction vessel, wherein the precursor is added to maintain a constant precursor concentration throughout the entirety of the reaction.

Most enzymes are inhibited by the products that they form, and if these inhibitors can be removed and the reaction replenished then the catalyzing enzymes can retain activity for weeks or months. Glucosyltransferase enzymes such as SrUGT76G1 are relatively good biocatalysts and can achieve high concentrations of products compared to the substrate concentration, displaying an equilibrium constant (Keq) of greater than 20 in a static reaction. This reaction equilibrium of products to substrates can be improved upon through removal of the products from the reaction, allowing the enzyme to continue catalyzing the substrate to product conversion. For example, in the production of THC-glycosides and CBD-glycosides as disclosed herein, the primary biocatalyst, the enzyme SrUGT76G1 (UGT76G1 protein from Stevia rebaudiana), is quickly inhibited by its two products, THC-glycosides or CBD-glycosides and UDP. To address these reaction limitations, a UDP cofactor regeneration system that utilizes SrSUS1 is employed to break a sucrose molecule to re-charge UDP to UDPG. Further, the THC-glycosides are removed from the biocatalytic reaction mixture.

To remove the product from the biocatalytic reaction mixture, a single reactor system that incorporates filtration and product capture that can be run while the reaction is ongoing, is used. Selectively removing the product (e.g., THC-glycoside or CBD-glycoside) boosts overall yields and allows for a continuous long-term biocatalytic reaction that can run for months. To remove the product from the biocatalytic reaction mixture, the biocatalytic reaction mixture is filtered. The biocatalytic reaction mixture is filtered to achieve a filtration retentate and a filtration permeate. The filtration permeate comprises the product (e.g., cannabinoid-glycoside). The filtration permeate may also comprise UDP, UDPG, sucrose, fructose, Mg, and/or other small and polar molecules. The filtration retentate may comprise the cannabinoid aglycone, glycosyltransferase enzyme (UGT76G1), Uridine diphosphate glucose (UDP-glucose), Sucrose Synthase isoform 1 from Stevia rebaudiana (SrSUS1), sucrose, or any combination thereof The retentate fraction is returned to the first vessel to be used in continuing reactions.

The reaction occurs in an aqueous solution that is continuously filtered through a tangential flow filter. The filter may comprise a modified polyethersulfone (mPES) membrane having a pore size of 10 kDa. The enzymes SrUGT76G1 and SrSUS1 do not permeate the filter due to pore size. The cannabinoid (e.g., THC or CBD) does not permeate the filter due to the mPES membrane chemistry. Cannabinoids are hydrophobic and non-polar while the mPES membrane is hydrophilic and polar. As a result, the cannabinoid is rejected by the filter and is retained in the filtration retentate. Cannabinoid-glycosides (such as THC- and CBD-glycosides) are considerably more hydrophilic and polar than the cannabinoid itself, allowing the cannabinoid-glycosides to pass freely through the mPES filter and thus comprise the filtration permeate. Standard PES filters also allow the cannabinoid-glycosides to pass freely through the PES filter, but also allow more of the cannabinoid to pass through as well.

Biocatalytic reactions typically employ expensive recombinant enzymes as the catalysts, so utilization of the full production potential of the enzyme is critical to minimize its cost relative to the product. Tangential flow filtration and reverse phase chromatography are commonly referred to as “Downstream Processing” technologies and are typically used to process a terminally produced mixture “downstream” or after the main reaction. This disclosure combines the downstream processes into the reaction itself, as the cannabinoid-glycosides are filtered and purified while the reaction is running.

The filtration permeate which passes through the filter is flowed directly onto a packed chromatography bed. In some instances, such as when making cannabinoid-glycosides, the packed chromatography bed is C18 fused/functionalized to silica. Cannabinoid-glycosides are captured on the C18 silica while other more water soluble compounds pass through (the permeate return) and are returned to the reaction. The reaction can operate continuously for over 500 hours with only the cannabinoid (e.g., THC or CBD) as input. FIGS. 1-3 show an overview schematic for the process and reactor system, and more detailed schematics for the components in the continuous perfusion reactor system.

Enzymes are biological catalysts, and chemical reactions are typically faster at higher temperatures. This phenomenon is widely known and is exemplified by the Arrhenius equation for reaction kinetics. Enzymes from non-thermophiles exhibit kinetic rates proportional to increased temperatures, but unlike inorganic catalysts, enzymes suffer from secondary structure disruption and unfolding at high temperatures, resulting in a loss of catalytic activity. As such, it will be appreciated by those skilled in the art, that the temperature of the reaction affects the reaction rate and enzymatic yields. The temperature can be adjusted depending on components of the system and the desired product.

For example, the reaction catalysts utilized to produce cannabidiol-glycoside were tested at multiple reaction temperatures from 20° C. to 50° C. At higher temperatures the enzyme activity was shorter lived. For example, the optimal temperature for the reaction activity balanced with longevity of the enzyme for the production of cannabinoid-glycosides (e.g., CBD- and THC-glycosides) was found to be about 44° C.

In some embodiments, the reaction temperature may be about 20° C. to about 50° C. or about 30° C. to about 40° C. In some embodiments, the reaction temperature may be about 40° C. to about 50° C. In some embodiments, the reaction temperature may be about 42° C. to about 46° C. In some embodiments, the reaction temperature may be about 40° C., about 41° C., about 42° C., about 43° C., about 44° C., about 45° C., about 46° C., about 47° C., about 48° C., about 49° C., or about 45° C. In some embodiments, the reaction temperature may be about 44° C. For example, when making THC-glycosides or CBD-glycosides, the reaction temperature may be about 44° C. The temperature of the reaction may be controlled in the main reactor vessel by a jacket heater or similar.

As discussed, in some embodiments, the disclosed process may be used for the production of one or more cannabinoid-glycosides. The cannabinoid-glycoside may be selected from one or more of the cannabinoid-glycosides disclosed in WO 2017/053574 (PCT/US2016/053122) and/or WO 2021/173190 (PCT/US2020/19886), the entirety of which are incorporated by reference. The cannabinoid glycosides may be THC-glycosides. The cannabinoid-glycosides maybe CBD-glycosides.

In some embodiments, the cannabinoid-glycoside is a THC-glycoside. The THC-glycoside may be selected from one or more of:

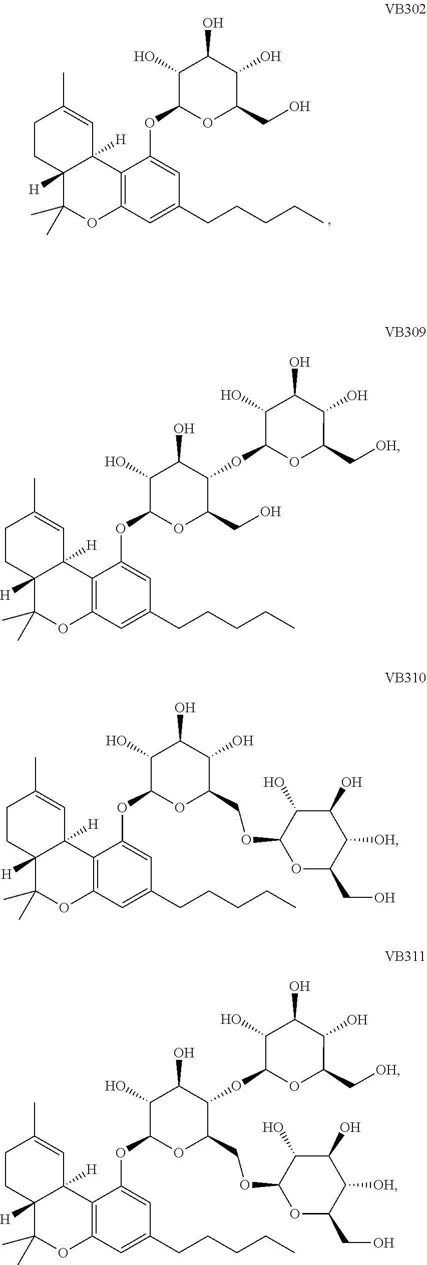

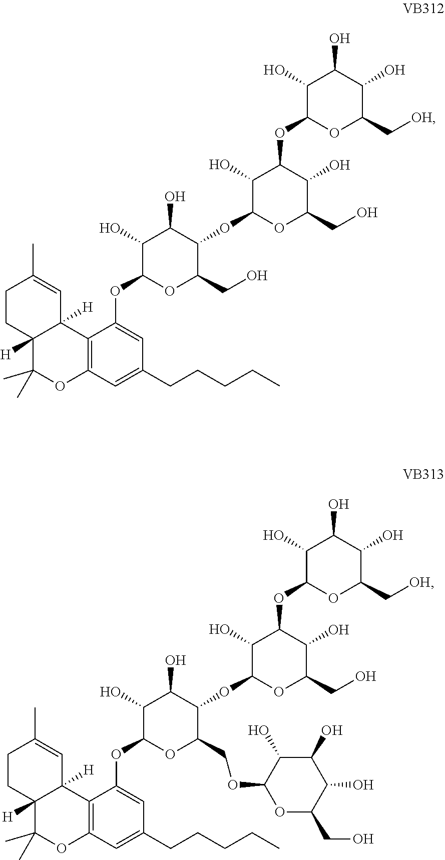 [SMILES: CCCCCc1cc(O[C@@H]2O[C@H](CO)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21 ; CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

or any combination thereof.

In some embodiments, the cannabinoid-glycoside is a CBD-glycoside. The CBD-glycoside may be selected from one or more of:

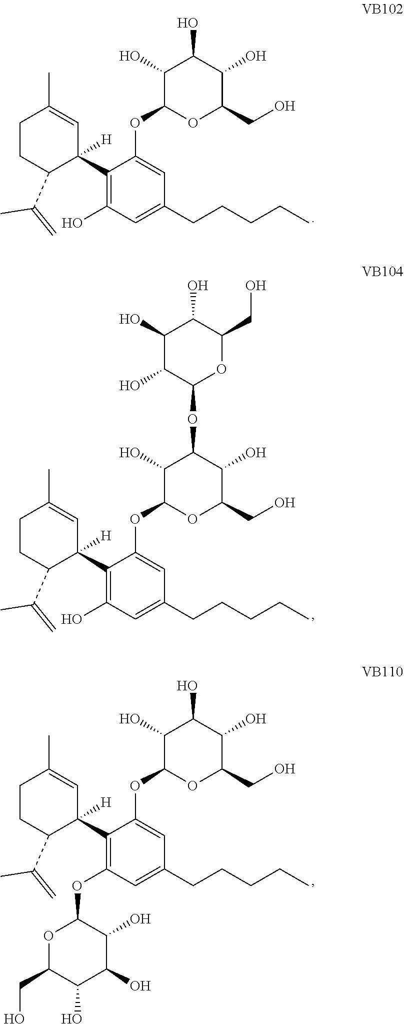

or any combination thereof.

It will be appreciated by those of skill in the art that the disclosures can be adapted for the production of other molecules and are not limited to the production of cannabinoid-glycosides.

The present disclosures also relates to reactor systems and apparatus for the production of hydrophobic product. The disclosed reactor systems have the benefit of allowing for efficient continuous production of the end product. Further, the disclosed reactor systems can be used to perform the processes previously disclosed herein.

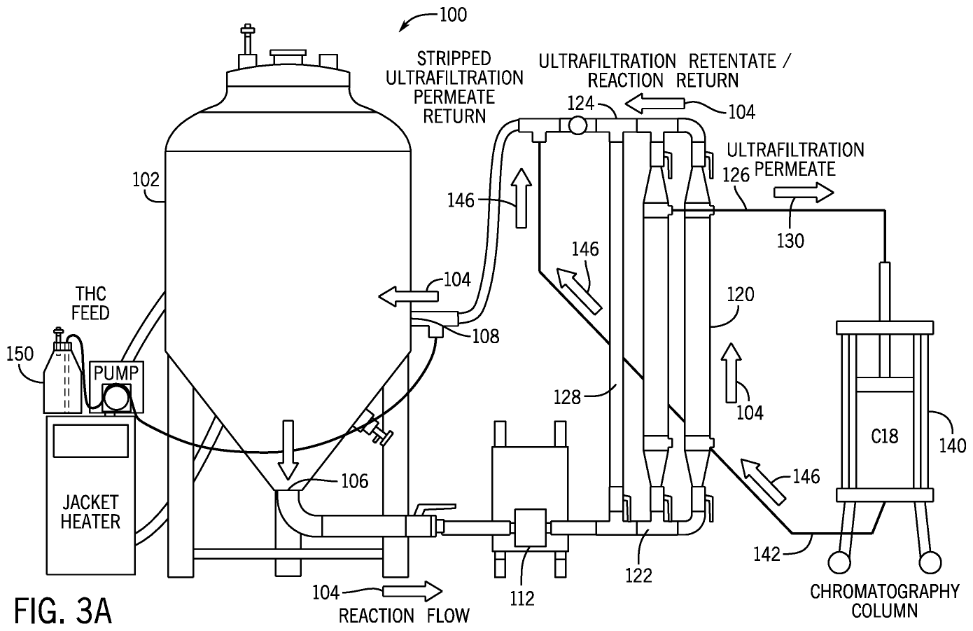

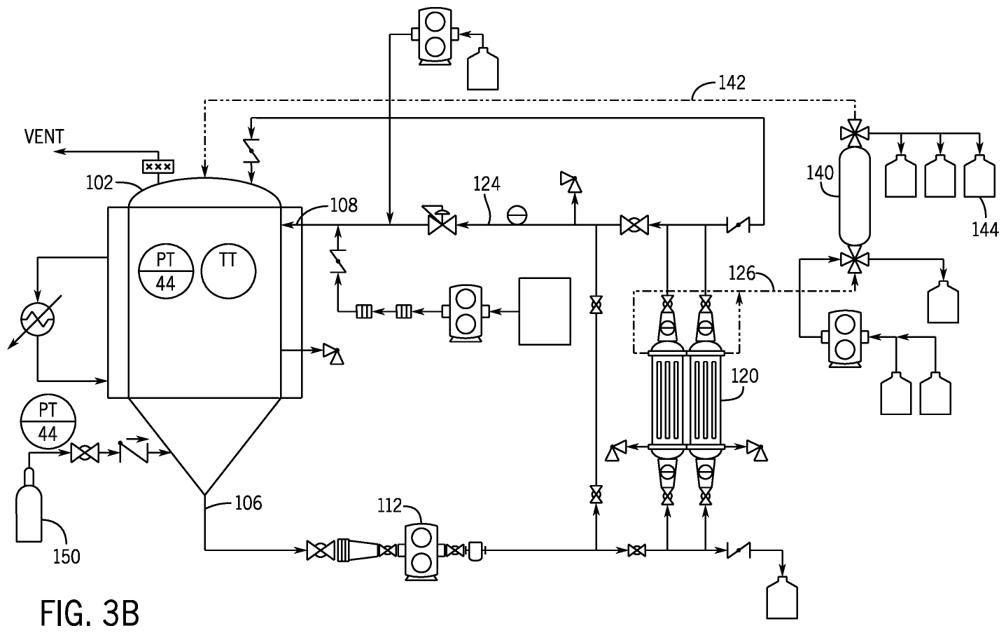

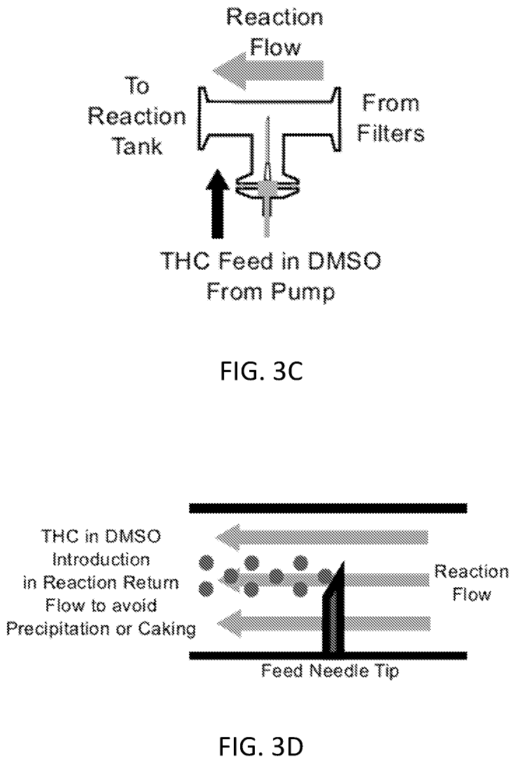

Referring to FIGS. 3A-3B, non-limiting schematics of a continuous reactor system 100 defining a reaction flow path illustrated by arrows 104 are provided. The reaction system 100 includes a reaction vessel 102 having an outlet 106 for flow from the vessel 102 and a return 108 for return to the vessel 102. Flow is provided via pump 112, which may be a low-shear lobe pump; however, other pumping systems may be used in the continuous reactor system.

The reaction vessel 102 is illustrated as a cylindroconical vessel. However, other suitably designed reaction vessels are within the scope of the present disclosure. The reaction vessel 102 may be made from stainless steel or other suitable materials. The conical nature of the reaction vessel 102 and continuous pumping by pump 112 are used to mix the reaction. The conical vessel 102 is designed to provide mixing without the need for an impeller. In addition, the conical vessel 102 provides for consistent turbulence in the fluid flow, void of non-turbulent spaces or surfaces for materials to settle out of the reaction. In other systems, however, other systems of mixing vessels may be employed.

From the vessel 102, an aqueous solution is pumped along the flow path 104 through a filtration system 120. In the illustrated embodiment the filtration system 120 is shown as an array of hollow fiber mPES membranes by a low-shear lobe pump. The filtration system 120 has an inlet 122 and a reaction return outlet 124. The reaction return outlet 124 returns the filter retentate to the reaction flow path 104 and ultimately to the reaction vessel 102. In addition, the filtration system 120 includes a product outlet 126 for ultrafiltration permeate, with flow illustrated by arrow 130. In addition, the schematic includes a bypass line 128 for bypassing the filtration system 120.

The filter permeate from the product outlet 126 of the filtration system 120, with flow illustrated by arrow 130, is then passed through a C18 chromatography column 140. The unbound permeate is returned to the reaction flow path 104 and ultimately to the reaction vessel 102 via flow line 142 with flow illustrated by arrow 146. Therefore, the chromatography column 140 removes only the reaction products 144.

In accordance with some embodiments of the present disclosure, a reactor system 100 may comprise (a) a main reactor vessel 102; (b) a substrate feed system 150; (c) a tangential-flow filtration system 120; and (d) a chromatography column 140, with the filtration system 120 and the chromatography column 140 in a dual nested loop configuration (see FIG. 1). The reactor system may also include additional components and elements as shown in FIGS. 3A-3B or as will be appreciated by those of skill in the art (such as vents, sensors, valves, etc.).

In some embodiments, the substrate solution may be fed through a needle point see, FIGS. 3C-3D) that is submerged in the turbulent retentate return flow path just before it enters the vessel 102 (see, FIG. 3A), encouraging quick dispersion and solubility of the precursor to the product (e.g., the cannabinoid such as THC or CBD). In some instances, high concentrations of the precursor to the product can lead to substrate precipitation in the aqueous reaction mixture. Once precipitated, the precursor to the product (e.g., THC, CBD, etc.) coats the surfaces and crevices inside the reaction system, which has a cascade effect in that it is drawn out of solution faster, compounding the problem. To solve this problem, the substrate solution is fed through a needle point (see, FIGS. 3C-3D) that is submerged in the turbulent retentate return flow path 104 just before it enters the vessel 102 (reaction tank) at the return 108 (see, FIG. 3A), encouraging quick dispersion and solubility of the precursor to the product (e.g., THC, CBD, etc.).

The disclosed reactor system may be run continuously or semi-continuously. The reactor system may be run for days, weeks, or months.

It will be appreciated by those of skill in the art that the disclosed reactor systems may be used for the production of a wide variety of molecules including those described herein. For example, the reactor system may be used for the production of glycosides. The glycosides may be cannabinoid-glycosides. In some embodiments, the cannabinoid-glycosides may be any of those disclosed herein.

Further, a simplified reaction with only sucrose synthase enzyme may be used with a suitable phosphate-rejecting membrane chemistry for the production of UDPG. Potential phosphate-rejecting membrane chemistry may include a negatively charged surface chemistry, to reject the UDP but allow the UDPG to pass.

As various changes could be made in the above described processes and systems without departing from the scope of the present disclosure, it is intended that all matter contained in the above description, shall be interpreted as illustrative and not in a limiting sense.

The following examples are offered by way of illustration and not by way of limitation.

### EXAMPLE 1

### Enzymatic Perfusion Reactor System and Process

An exemplary process of producing hydrophobic products is shown in FIG. 1. A biocatalytic reaction occurs in a reactor (box 12). The reaction is then pumped to hollow-fiber tangential-flow ultrafiltration means box 20), where it is filtered with a majority of the reaction returning to the reactor (box 40). The small amount of the reaction that permeates through the filtration means such as a filter is pumped to C18 columns (box 3), wherein reaction products are bound to the C18 chromatography resin, and unbound materials are returned to the reaction. The reaction is stripped of products that would inhibit the biocatalyst, freeing up further catalysis of more product while also acting as an intra-reaction processing step. FIG. 1 illustrates the nested loops of the process.

### EXAMPLE 2

### THC Glycosylation Reactions

Δ9-tetrahydrocannabinod (THC)-glycosides can be produced in an aqueous solution using a biocatalytic reaction to create a biocatalytic reaction mixture. For the biocatalytic reaction, one or more reagents may be involved, such as THC, a glycosyltransferase enzyme, Uridine diphosphate glucose (UDP-glucose), a sucrose synthase, sucrose, and any combination thereof. The sucrose synthase may be one or more of isoforms 1 through 6 and may be derived from Arabidopsis thaliana or Stevia rebaudiana. For example, the sucrose synthase is isoform 1 derived from Stevia rebaudiana (SrSUS1). As shown in FIG. 2, a recombinant glycosyltransferase enzyme (SrUGT76G1) covalently links glucose molecules to THC in a biocatalytic reaction. Uridine diphosphate glucose (UDP-glucose) acts as the sugar donor and is regenerated within the reaction using a second recombinant enzyme, sucrose synthase (SrSUS1). SrSUS1 catalyzes the splitting of 1M sucrose to fructose and glucose to recycle UDP into UDP-glucose.

The biocatalytic reaction may occur in a continuous perfusion bioreactor as shown in FIG. 3A. The reaction takes place in the large cylindroconical stainless steel vessel 102 and is pumped through the hollow fiber mPES membranes 120 by a low-shear lobe pump or positive displacement low-shear pump. The conical nature of the vessel and the constant pumping are used to mix the reaction. There is no impeller on the vessel, and the conical vessel doesn't have non-turbulent spaces or surfaces for materials to settle out of the reaction. However, in some embodiments, an impeller may be optionally included on the vessel to increase mixing. The filter permeate is then passed through a C18 chromatography column 140, and both the filter retentate and unbound permeate are returned to the reaction, removing only the reaction products. Another technical reactor schematic illustrating the main reactor and supporting systems is shown in FIG. 3B.

As illustrated in FIGS. 3A-3B, a continuous perfusion bioreactor may comprise a main reactor vessel; a substrate feed system; a tangential-flow filtration system; and a chromatography column, in a dual nested loop configuration. The reactor system may also comprise additional components. The temperature of the reaction may be controlled in the main reactor vessel by a jacket heater or similar.

The reaction products such as THC-glycoside products can be removed from the biocatalytic reaction mixture using the reactor described above. In this process, the biocatalytic reaction mixture is filtered to achieve a filtration retentate and a filtration permeate. The filtration permeate comprises the THC-glycoside. The filtration retentate may comprise THC, glycosyltransferase enzyme (UGT76G1), Uridine diphosphate glucose (UDP-glucose), Sucrose Synthase isoform 1 from Stevia rebaudiana (SrSUS1), sucrose, and any combination thereof. The retentate fraction is returned to the vessel 102 to be used in continuing reactions.

The reaction occurs in an aqueous solution that is continuously filtered through filtration means such as a tangential flow filter. The filter may comprise a modified polyethersulfone (mPES) membrane 120 having a pore size of 10 kDa. The enzymes SrUGT76G1 and SrSUS1 do not permeate the filter due to pore size. THC does not permeate the filter due to the mPES membrane chemistry. THC is hydrophobic and non-polar while the mPES membrane is hydrophilic and polar. As a result, THC is rejected by the filter and is retained in the filtration retentate. THC-glycosides are considerably more hydrophilic and polar than THC, allowing them to pass freely through the mPES filter and thus comprise the filtration permeate to be captured by the C18 chromatography column 140.

The concentration of the hydrophobic THC substrate needs to be closely monitored. High concentrations of THC inhibit SrUGT76G1 and can also lead to substrate precipitation in the aqueous reaction mixture. Once precipitated, THC coats the surfaces and crevices inside the reaction system, which has a cascade effect in that it is drawn out of solution faster, compounding the problem. A drastic decrease in reaction productivity is observed once THC precipitates. To solve this problem, the substrate solution is fed through a needle point (see, FIGS. 3C-3D) that is submerged in the turbulent retentate return flow path just before it enters the vessel 104 (see, FIG. 3A), encouraging quick dispersion and solubility of the THC.

The THC substrate was dissolved into DMSO and fed from a 2 L or 5 L glass media bottle. A ¼″ NPT hole is tapped into the lid where a luer lock+NPT+barb fitting was attached. A dip tube draws feed from the bottom of the bottle through the lid fitting via peristaltic pump. On the other end of the pump, the feed hose terminates in a luer lock barb fitting and attaches to the needle injection port (FIGS. 3A, 3C). Two pinch valves, between the pump and needle port and between the pump and substrate feed bottle 150 were used to isolate the line and change feed bottles or pumps.

The needle injection port was constructed of an 18-gauge needle attached to a luer lock barb fitting and chemically resistant tubing that was snuggly pulled through a TC x hose barb fitting. The needle port attached to a tee on the retentate return line. The point of the needle was placed into the middle of the flow path of the retentate return line (see, FIG. 3C).

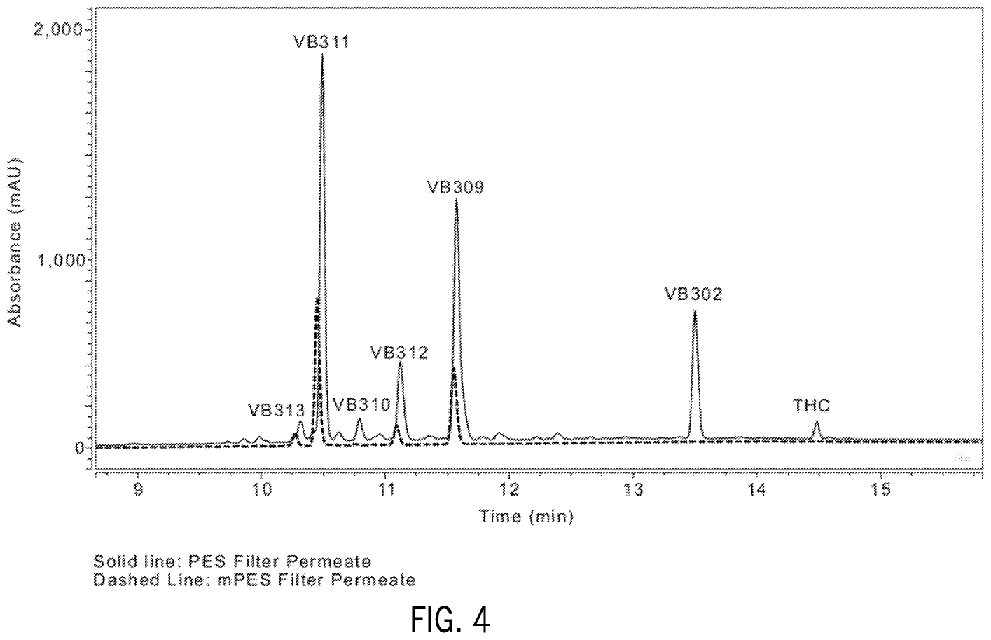

A HPLC linetrace comparing mPES filter chemistry rejection of THC and hydrophobic compounds is shown in FIG. 4. The solid line is the mPES filter retentate, which is representative of the whole reaction mixture including THC and hydrophobic VB302 (THC-glycoside). The dashed line is the mPES filter permeate, which is representative of the reaction that is able to pass through the mPES filter. The mPES filter permeate does not contain THC or VB302 but contains VB309, VB312, VB310, VB311, and VB313, supporting that mPES rejects hydrophobic THC and VB302 while allowing more polar THC-glycosides to pass through. That is, VB302 and THC are able to permeate through a PES membrane, but not through the modified PES (mPES) membrane chemistry. Less hydrophobic THC-glycoside products are able to permeate/pass through either membrane chemistry.

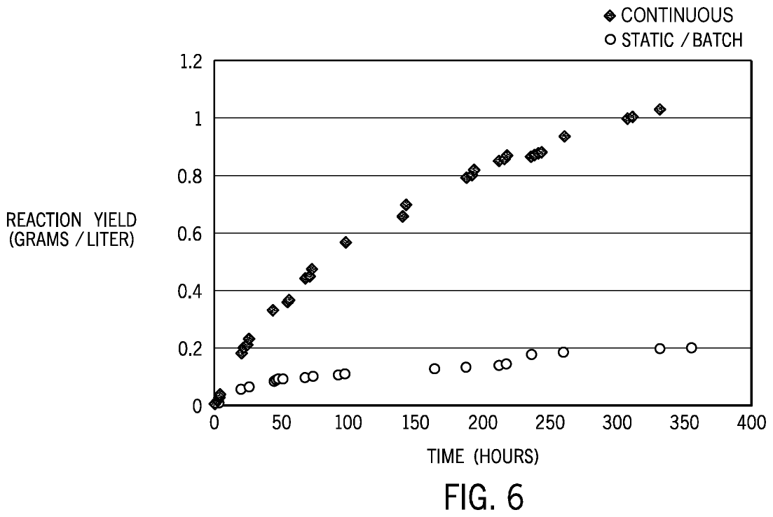

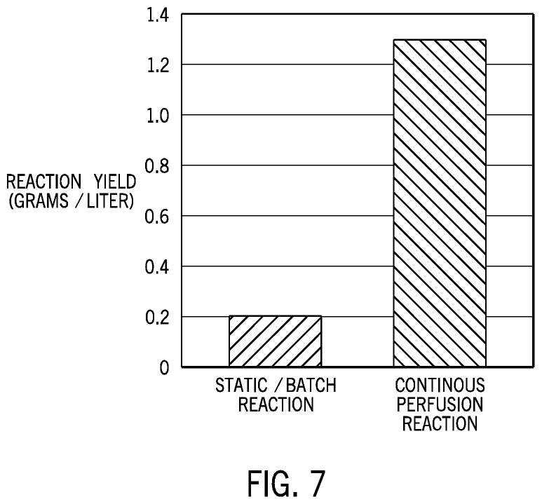

The continuous perfusion reaction process has an advantage of producing increased yields of THC-glycosides. In this process, a continuous biocatalytic reaction occurs in an enzymatic perfusion reactor, where THC is converted to glycosides of THC, which allows for increased yields, reduced waste/workload, and the ability to produce a compound that otherwise would not have been economically feasible. FIGS. 6-7 demonstrate the observed increased yields.

FIG. 5 shows product removal and reaction recovery cycles in the continuous perfusion reaction, which is depicted as reaction yield (g/L) over time (hours). The reaction was run in semi-continuous mode with intermittent product removal. The reaction was allowed to accumulate product without filtration until the concentration of product starts to inhibit the enzyme. Filtration and removal of the product was initiated and run until the product concentration was below a pre-determined threshold or at a product concentration conducive to enzyme activity. The reaction was terminated after the rate of product formation no longer met production specifications.

THC-glycoside products produced from SrUGT76G1/SrSUS1 continuous vs. static/batch biocatalytic reactions were compared and the results are shown in FIG. 6. A static/batch reaction (open circles) produced 0.2 g/L total THC-glycosides over the duration of the batch, whereas a continuous reaction (grey diamonds) produced over 1 g/L total THC-glycosides over the duration of the reaction. The comparison of the final THC-glycoside production in grams/Liter of static/batch vs. continuous perfusion reactions with SrUGT76G1 as the biocatalyst and similar reaction recipes is shown in FIG. 7. The final yield in the continuous perfusion reaction is significantly higher than the final yield in the static/batch reaction.

### EXAMPLE 3

### Substrate Feed

The retentate is sampled, extracted, and the substrate and product concentrations are quantified via HPLC. The retentate is sampled every hour after the substrate feed is first initiated. Once the THC concentration has stabilized and the appropriate feed rate has been determined, the reaction should be sampled at least 3 times a day. More frequent sampling may be required if the THC concentration in the reaction is fluctuating outside the accepted range (3-5 mg/L). The operator of the feed should determine how often and when the retentate needs to be sampled to keep the reaction in check.

The lag time between sampling the reaction and being able to analyze the HPLC data is around 1 hour. Without real-time THC concentration data, it can be difficult to make accurate THC feed rate estimations. In addition, long durations where the reaction cannot be sampled (overnights and weekends) creates lapses in the sampling data. A long period with an incorrect feed rate can reduce the overall productivity of a reaction.

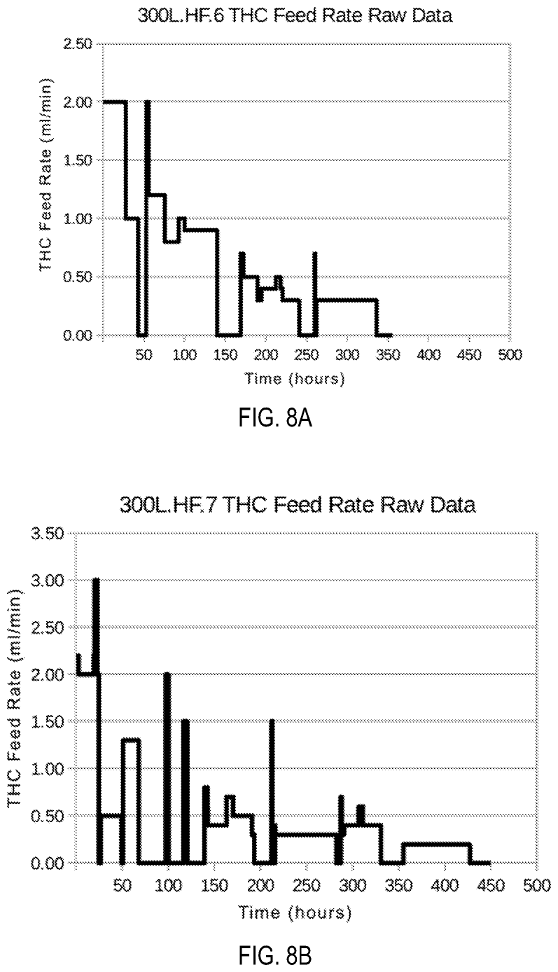

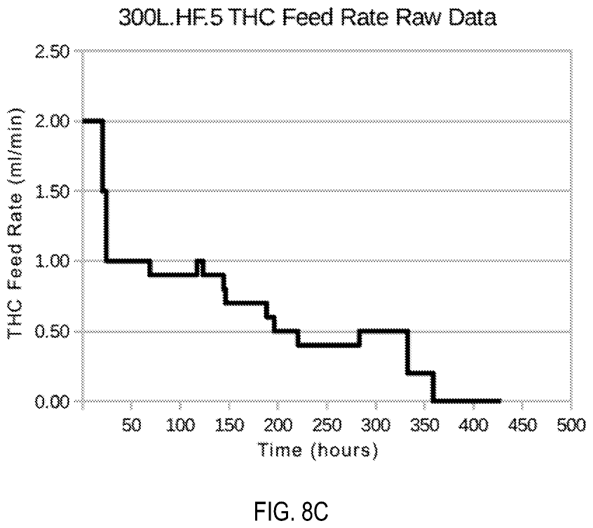

THC feed rate estimation: A general guideline for the rate of THC addition is provided below based on the review of three 300 L reactions in which the feed rate was empirically determined (FIGS. 8A-8C). The rate of THC consumption was measured by stopping the THC feed and quantifying the drop in substrate concentration over a given period. The rate of feed required was estimated and then adjusted when HPLC data indicated the THC concentration was outside the accepted range (3-5 mg/L). The feed solution is THC is in DMSO at a concentration of 12 mg/mL.

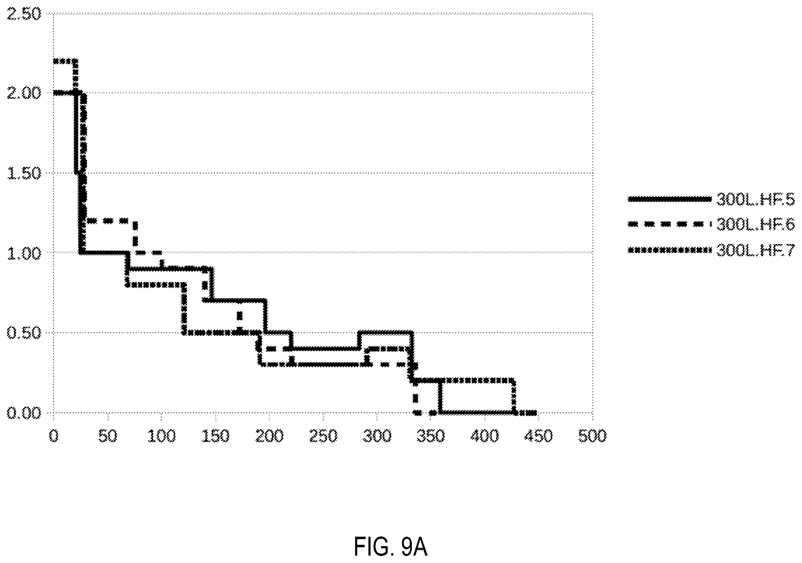

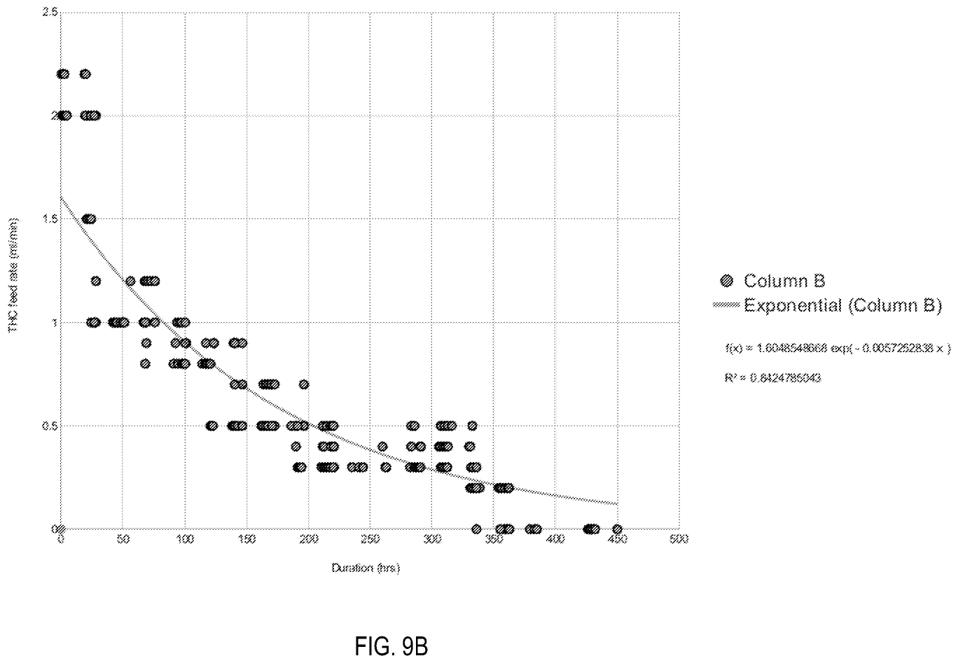

The THC feed rate data was normalized by removing the extreme adjustments to the feed. FIG. 9A shows the corrected THC feed rates of the three reactions after all the zero feed rates and inaccurately high feed rate time frames have been changed to reflect the stable feed rate prior to and post feed adjustments. FIG. 9B shows the three data sets combined and fit as an exponential decay to provide a guideline for the expected feed rate over the course of the reaction.

The expected rate of THC feed for a 300 L reaction is given in Table 1 below. The rate of THC consumption in milligrams per liter of reaction volume is also given.

TABLE 1
Expected THC Feed Rate (12 mg/mL in DMSO)

| Timepoint (hrs) | ml/min of feed | mg/L of reaction/hr |
|---|---|---|
| 0.0 | 2.00 | 4.8 |
| 32 | 1.5 | 3.6 |
| 75 | 1.12 | 2.69 |
| 100 | 1.00 | 2.4 |
| 150 | 0.72 | 1.73 |
| 200 | 0.53 | 1.27 |
| 250 | 0.39 | 0.94 |
| 300 | 0.3 | 0.72 |
| 350 | 0.2 | 0.48 |
| 400 | 0.15 | 0.36 |

### EXAMPLE 4

### CBD Fed-Batch Glycosylation Reactions

Scale and relative productivity of CBD fed-batch glycosylation reactions were compared to THC fed reactions. CBD reactions were developed at the 200 μl scale to determine the optimum UDP, SUS1 & 76G1 concentrations, the optimum CBD seed concentration and finally the optimized reaction kinetics. The optimized reaction conditions were determined to be 1.5 mM UDP input, 3% SUS1 and 10% 76G1, with a CBD seed concentration of 0.01 mg/mL.

Next, the optimized CBD reaction was scaled up to a 1 L static batch reaction to determine if the reaction kinetics scaled linearly. The reaction proceeded 50% faster at the 1 L scale, but this is attributed to much more efficient stirring/agitation of the reaction mixture. The 1 L reaction consumed 20 mg/hr CBD. A total of 145 mg of CBD was dosed into the reaction and the CBD glycoside reaction yield was 362.5 mg total for a 2.5× input to product mass inflation.

A second 1 L CBD reaction was performed but this time in a fed batch manner in order to optimize a continuous feed rate. 20 mg/hr CBD was fed into the reaction mixture and reaction kinetics were maintained until between hour 3 and 4 when product inhibition was observed, indicating the need to initiate a reaction scrub. The continuous feed rate was dropped to 5 mg/hr for the remainder of the reaction. A total of 600 mg of CBD was fed into the reaction and the CBD glycoside reaction yield was 1.2582 g for a total of a 2.1× input to product mass inflation.

Finally, the reaction was scaled to 10 L fed batch reaction. The feed rate scaled linearly as the 10 L reaction consumed 200 mg/hr CBD until between hours 3 and 4, as observed in the 1 L fed batch reaction indicating product inhibition. A reaction scrub was initiated at 4.25 hours at a pump speed of 50 ml/min through the hollow fiber module, and the feed was dropped to 48 mg/hr. This scrub and feed rate maintained a stable CBD concentration in the reaction mixture indicating that this is optimum for running the CBD reaction in a truly continuous manner. A total of 3.0 g of CBD input was fed into the reaction and a total of 7.8395 g of CBD-glycosides (referred to as VB100X) was yielded, for an input to product mass inflation of 2.61×.

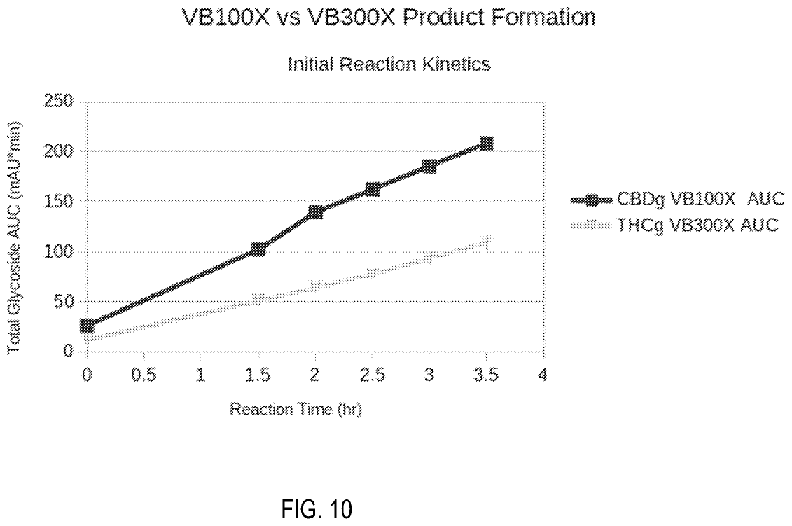

When comparing the efficiency of a 10 L CBD to a 300 L THC conversion reaction, 47.7% more VB100X is formed in the first 3.5 hours the reaction compared to VB300X (see, FIG. 10). Product scrubbing is initiated within the first 4-5 hours during a CBD reaction as product is formed very quickly, whereas it is initiated at approximately 36 hours for a THC conversion reaction.

The CBD was far more active than THC, so it needed to be “scrubbed” from the reaction far more frequently than THC (meaning the pump was turned on to run the reaction through the mPES filter and the CBD-glycoside mixture was bound by the C18 chromatography column). The CBD reaction was scaled to 10 L, and scaling the reaction from 100 μL to 10 L represents a 100,000× reaction scale-up.

Although the present disclosure and its advantages have been described in detail, it should be understood that various changes, substitutions and alterations can be made herein without departing from the spirit and scope of the disclosure as defined in the appended claims.

One skilled in the art will readily appreciate that the present disclosure is well adapted to carry out the objects and obtain the ends and advantages mentioned, as well as those inherent therein. The present examples along with the methods described herein are presently representative of preferred embodiments, are exemplary, and are not intended as limitations on the scope of the disclosure. Changes therein and other uses will occur to those skilled in the art which are encompassed within the spirit of the disclosure as defined by the scope of the claims.

1. A method for the production of a cannabinoid-glycoside comprising:
(a) producing the cannabinoid-glycoside in an aqueous solution to obtain a biocatalytic reaction mixture;
(b) filtering the biocatalytic reaction mixture comprising the cannabinoid-glycoside to achieve a filtration retentate and a filtration permeate comprising the cannabinoid-glycoside;
(c) returning the filtration retentate to a first vessel;
(d) separating the cannabinoid-glycosides from the filtration permeate using reverse-phase chromatography such that the cannabinoid-glycosides bind to the chromatography resin; and
(e) returning unbound filtration permeate to the first vessel.

2. The method of claim 1, wherein the cannabinoid-glycoside is a Δ9-tetrahydrocannabinol (THC)-glycoside, a cannabidiol (CBD)-glycoside, or any combination thereof.

3. The method of claim 2, wherein the method further comprises adding THC and/or CBD and one or more reagents to the first vessel.

4. (canceled)

5. The method of claim 3, wherein the one or more reagents comprise glycosyltransferase enzyme (UGT76G1), Uridine diphosphate glucose (UDP-glucose), Sucrose Synthase (SUS), sucrose, and any combination thereof.

6. The method of claim 5, wherein the Sucrose Synthase is isoform 1.

7. The method of claim 6, wherein the Sucrose Synthase isoform 1 is from Stevia rebaudiana (SrSUS1).

8. The method of claim 1, wherein the filtering comprises modified PES (mPES), hollow-fiber tangential-flow ultrafiltration, and/or a spiral-wound configuration.

9. (canceled)

10. (canceled)

11. The method of claim 1, wherein the method further comprises collecting the cannabinoid-glycosides from the chromatography resin.

12. (canceled)

13. The method of claim 2, further comprising adding THC and/or CBD to the first reaction vessel, wherein the THC or CBD is added to maintain a constant THC or CBD concentration throughout the entirety of the reaction.

14. The method of claim 1, wherein the method can be run continuously or semi-continuously for days, weeks, or months.

15. A system for the production of a hydrophobic product, the system comprising:
a reaction vessel having a vessel outlet and a vessel return;
a reaction flow path extending from the vessel outlet to the vessel return;
a precursor of the product to the vessel or the reaction flow path; and
a filtering system disposed within the reaction flow path for separating products from the reaction flow path to achieve a filtration retentate and a filtration permeate, wherein the filtration permeate includes the product, the filtering system having a filter inlet, a filter return to return the filtration retentate to the reaction flow path, and a permeate outlet for the filtration permeate.

16. The system of claim 15, further comprising a chromatography system for separating the product from the filtration permeate using reverse-phase chromatography such that the product binds to the chromatography resin.

17. The system of claim 16, wherein the chromatography system further comprises a chromatography return to the vessel or the reaction flow path for unbound filtration permeate.

18. The system of claim 15, wherein the system is continuous or semi-continuous.

19. The system of claim 15, wherein the filtering system comprises a hollow fiber PES membrane filter.

20. The system of claim 15, wherein the reaction vessel is an enzymatic perfusion reactor.

21. The system of claim 15, wherein the product is a Δ9-tetrahydrocannabinol (THC)-glycoside, a cannabidiol (CBD)-glycoside, or any combination thereof.

22. (canceled)

23. A process for the production of a hydrophobic product, the process comprising:
(a) mixing a biocatalytic reaction mixture in a reaction system including a reaction vessel having a vessel outlet and a vessel return and a reaction flow path extending from the vessel outlet to the vessel return;
(b) along the reaction flow path, filtering the biocatalytic reaction mixture to produce a filtration retentate and a filtration permeate, wherein the filtration permeate comprises the product; and
(c) returning the filtration retentate to the reaction vessel.

24. The process of claim 23, further comprising separating the product from the filtration permeate using reverse-phase chromatography such that the product binds to the chromatography resin.

25.-28. (canceled)

29. The process of claim 23, wherein the product is a Δ9-tetrahydrocannabinol (THC)-glycoside, a cannabidiol (CBD)-glycoside, or any combination thereof.
